SEQUENCE LISTING (3 sequences)

SEQ ID NO: 1 - DNA, 813 bp, synthetic construct
  source 1..813: mol_type = other DNA
tgcggaccttttaaagcttgcttttaaaaggttagcggcggacgggtgagtaacacgtgggcaacctgcctgtaagatcgggataacgccgggaaaccggggctaataccggatagttttttcctccgcatggaggaaaaaggaaagacggcttcggctgtcacttacagatgggcccgcggcgcattagctagttggtggggtaacggctcaccaaggcaacgatgcgtagccgacctgagagggtgatcggccacattgggactgagacacggcccaaactcctacgggaggcagcagtagggaatcttccgcaatggacgaaagtctgacggagcaacgccgcgtgagtgaagaaggccttcgggtcgtaaaactctgttgccggggaagaacaagtgccgttcgaacagggcggcgccttgacggtacccggccagaaagccacggctaactacgtgccagcagccgcggtaatacgtaggtggcaagcgttgtccggaattattgggcgtaaagcgcgcgcaggcggcttcttaagtctgatgtgaaatcttgcggctcaaccgcaagcggtcattggaaactgggaggcttgagtgcagaagaggagagtggaattccacgtgtagcggtgaaatgcgtagagatgtggaggaacaccagtggcgaaggcggctctctggtctgtaactgacgctgaggcgcgaaagcgtggggagcaaacaggattagataccctggtagtccacgccgtaaacgatgagtgctaagtgttagagggtttccgccctttagtgctgcagctaacgc

SEQ ID NO: 2 - DNA, 15 bp, synthetic construct
  source 1..15: mol_type = other DNA
tacggraggcagcag

SEQ ID NO: 3 - DNA, 20 bp, synthetic construct
  source 1..20: mol_type = other DNA
ggactachvgggtwtctaat

(19) United States
(12) Patent Application Publication
(10) Pub. No.: US 2024/0366689 A1
(43) Pub. Date: Nov. 7, 2024

(54) APPLICATION OF BACILLUS COAGULANS IN PREPARING MEDICATION FOR IMPROVING FUNCTIONAL DECLINE OF ORGANISMS CAUSED BY HYPERGLYCEMIA AND HYPERLIPIDEMIA

(71) Applicant: Thankcome Biological Science and Technology (Suzhou) Co., Ltd., Suzhou (CN)

(72) Inventors: Yang YU, Suzhou (CN); Xin MA, Suzhou (CN); Xueping YU, Suzhou (CN)

(21) Appl. No.: 18/511,300

(22) Filed: Jan. 29, 2024

Related U.S. Application Data
(63) Continuation of application No. PCT/CN2022/143689, filed on Jan. 16, 2023.

(30) Foreign Application Priority Data
Jan. 28, 2022 (CN) 202210105622.5

(51) Int. Cl. A61K 35/742; A61P 1/16; A61P 3/04; A61P 25/28; A61P 29/00

(52) U.S. Cl. CPC A61K 35/742; A61P 1/16; A61P 3/04; A61P 25/28; A61P 29/00

(57) ABSTRACT

An application of Bacillus coagulans in preparing a medication for improving functional decline of organisms caused by hyperglycemia and hyperlipidemia is provided in the present application, belonging to the technical field of microbial strains. A Bacillus coagulans is disclosed in the present application, and the Bacillus coagulans is preserved in the China General Microbiological Culture Collection Center (CGMCC), Institute of Microbiology Chinese Academy of Sciences on Nov. 10, 2021, with a preservation number of CGMCC No.23766 and a preservation address of Institute of Microbiology, Chinese Academy of Sciences, No.3, No.1 Beichen West Road, Chaoyang District, Beijing.

### CROSS-REFERENCE TO RELATED APPLICATIONS

This application is a continuation of PCT/CN2022/143689, filed on Jan. 16, 2023 and claims priority to Chinese Patent Application No. 202210105622.5, filed on Jan. 28, 2022, the entire contents of which are incorporated herein by reference.

### INCORPORATION BY REFERENCE STATEMENT

This statement, made under Rules 77(b)(5)(ii) and any other applicable rule incorporates into the present specification of an XML file for a “Sequence Listing XML” (see Rule 831(a)), submitted via the USPTO patent electronic filing system or on one or more read-only optical discs (see Rule 1.52(e)(8)), identifying the names of each file, the date of creation of each file, and the size of each file in bytes as follows:
File name: 20231031_sequence_347-052_2602
Creation date: Oct. 31, 2023
Byte size: 4,839

### TECHNICAL FIELD

The present application relates to the technical field of microbial strains, and in particular to an application of Bacillus coagulans in preparing a medication for improving functional decline of organisms caused by hyperglycemia and hyperlipidemia.

### BACKGROUND

Under normal circumstances, blood glucose and blood lipids are kept in balance by hormonal and neurological regulation, but due to a combination of genetic and environmental factors, the regulation of both systems is disturbed, resulting in hyperlipidemia and hyperglycemia, which are commonly referred to as two of the “three highs (hyperlipidemia, hyperglycemia and hypertension)”. In addition to hyperlipidemia and hyperglycemia, many other diseases are associated with excessive blood glucose and lipids, including obesity, coronary heart disease, as well as declining in many physical functions. More importantly, the diseases caused by hyperglycaemia and hyperlipidaemia are taking place at a younger age, which has raised much attention, and it is of great practical importance to control hyperglycaemia and hyperlipidaemia and the various diseases they cause, especially the decline of physical functions.

Probiotics are live microorganisms beneficial to the host organisms and are useful in preventing various diseases when consumed in certain doses. Different strains offer universal functions among species and respective unique functions under combined influence of genetic factors and environmental factors. Therefore, it is necessary to continuously develop new beneficial bacteria strains so as to screen new medications against diseases and declining of body functions caused by hyperlipidemia and hyperglycemia by using the functions of the bacteria as well as the specific functions different from various species or strains of the same species.

### SUMMARY

The objectives of the present application include providing an application of a Bacillus coagulans in preparing a medication for improving functional decline of organisms caused by hyperglycemia and hyperlipidemia, so as to solve the problems existing in the prior art. The Bacillus coagulans has a certain effect on relieving weight gaining, inflammatory reaction, tissue fat accumulation, memory loss, intestinal short-chain fatty acid reduction and intestinal flora imbalance caused by high-sugar and high-fat diet, and provide new materials and directions for screening medications with efficacy of regulating functional decline of organisms caused by hyperlipidemia and hyperglycemia.

In order to achieve the above objectives, the present application provides the following technical schemes:

a Bacillus coagulans, and the Bacillus coagulans has been preserved in China General Microbiological Culture Collection Center (CGMCC), Institute of Microbiology Chinese Academy of Sciences on Nov. 10, 2021, with a preservation number of CGMCC No. 23766 and a preservation address of Institute of Microbiology, Chinese Academy of Sciences, No. 3, No. 1 Beichen West Road, Chaoyang District, Beijing City, P.R. China.

The present application also provides a medication with efficacy of relieving hyperglycemia and hyperlipidemia, with the Bacillus coagulans or secondary metabolites of the Bacillus coagulans being main active ingredients.

Optionally, the medication includes the Bacillus coagulans in a bacterial content of 3×10⁵ colony-forming unit (CFU/g).

The present application also provides an application of the Bacillus coagulans in preparing a medication with efficacy of relieving hyperglycemia and hyperlipidemia, and the medication has any one of functions illustrated as follows:
(1) reducing weight increased by high sugar and high fat;
(2) preventing inflammatory reaction caused by high sugar and high fat;
(3) preventing weight increasing in liver and epididymal fat caused by high sugar and high fat;
(4) alleviating declining of memory ability caused by high sugar and high fat;
(5) preventing short-chain fatty acids (SCFAs) decreasing caused by high sugar and high fat; and
(6) improving dysbiosis of intestinal flora caused by high sugar and high fat.

The present application achieves the following technical effects:

a new strain of Bacillus coagulans is screened, with experiments suggesting that the strain is effective in alleviating weight gaining, inflammation (TNF-α and IL-6 in blood), memory loss, tissue fat accumulation and intestinal SCFAs reduction caused by high sugar and high fat; in the experiments, the strain is used at a concentration of 3×10⁵ CFU/g to gavage mice fed with a high sugar and high fat diet, and the results in the prevention group is better than that of the treatment group; moreover, the sequencing results of 16S ribosomal ribonucleic acid (16S rRNA) gene of intestinal flora suggest that the mice fed by high-sugar and high-fat diet show an improved intestinal flora structure after supplemented with the Bacillus coagulans, and it is observed from the results of microbial community function prediction that the performance of metabolic pathway is closer to that of mice fed with conventional diet under the condition of supplementing high sugar and high fat diet with the Bacillus coagulans. As illustrated in the present application, the Bacillus coagulans has a modulating effect on the physiological metabolic changes associated with diabetes caused by a high sugar and high fat diet and the changed intestinal flora, therefore providing new materials and directions for improving intestinal flora and diseases, including diabetes, caused by high sugar and high fat.

### BRIEF DESCRIPTION OF THE DRAWINGS

For a clearer illustration of the technical schemes in the embodiments of the present application or in the prior art, a brief description of the accompanying drawings to be used in the embodiments are given below. It is obvious that the accompanying drawings in the following description are only some embodiments of the present application and that other accompanying drawings are available to those of ordinary skill in the art without any creative effort.

FIG. 1 shows weight changes in different groups.

FIG. 2 shows weight gaining in different groups.

FIG. 3 illustrates food intake of each mouse.

FIG. 4 illustrates energy intake of each mouse.

FIG. 5 shows levels of serum tumor necrosis factor (TNF-α) of mice at an end of an experiment.

FIG. 6 shows serum interleukin-6 (IL-6) levels of mice at the end of the experiment.

For the above statistics, data are expressed as mean±SEM, n=6; statistical differences are analyzed by one-way analysis of variance (ANOVA), with P<0.05 being statistically different; comparison of difference with normal feed diet (NFD) is expressed as #(#indicates P<0.05, ##indicates P<0.01, and ##indicates P<0.001), comparison of difference with HSD is expressed as * (* indicates P<0.05, ** indicates P<0.01, and *** indicates P<0.001), and ns stands for no significance.

FIG. 7 illustrates analysis of weight of mouse tissues (including liver, epididymal fat, and perirenal fat).

FIG. 8 shows results of hematoxylin-eosin (HE) staining of epididymal fat tissue.

FIG. 9 shows results of HE staining in liver tissue.

FIG. 10 illustrates result of oil red O staining in liver tissue.

FIG. 11 shows moving trajectory of mouse in a water maze.

FIG. 12 shows results of swimming time and distance of mouse in water maze experiment.

FIG. 13A shows changes of acetic acid in mice feces.

FIG. 13B illustrates changes of propionic acid in mice feces.

FIG. 13C shows changes of butyric acid in mice feces.

FIG. 14 shows results of Beta diversity analysis of microorganisms in mice feces.

FIG. 15 shows results of analysis of similarities (ANOSIM).

FIG. 16 shows results of microbial composition analysis of mice feces at phylum level.

FIG. 17A is abundance of Firmicutes in mice feces at the phylum level.

FIG. 17B is abundance of Bacteroidota in mice feces at the phylum level.

FIG. 17C is ratio of Firmicute/Bacteroidota.

FIG. 18 illustrates analysis results of microbial composition abundance of mice feces at family level.

FIG. 19 shows the microbial composition of mice feces in terms of abundance and ratio at family level.

FIG. 20 shows analysis results of microbial composition abundance of mice feces at genus level.

FIG. 21 shows the microbial composition of mouse feces in terms of abundance and ratio at genus level.

FIG. 22 is a microbiome heatmap of mice feces at genus level.

FIG. 23 shows a functional prediction heatmap (functional abundance statistics of clusters of orthologous groups (COG)) of phylogenetic investigation of communities by reconstruction of unobserved states (PICRUST) 2 at genus level.

FIG. 24 shows results of phenotypic prediction by BugBase.

### DETAILED DESCRIPTION OF THE EMBODIMENTS

Various exemplary embodiments of the present application are now described in detail and this detailed description should not be considered as limiting the present application, but should be understood as a rather detailed description of certain aspects, features and embodiments of the present application.

It should be understood that the terms described in the present application are intended to describe particular embodiments only and are not intended to limit the present application. Moreover, with respect to the range of values in the present application, it is to be understood that each intermediate value between the upper and lower limits of the range is also specifically disclosed. Each smaller range between any stated value or intermediate value within a stated range and any other stated value or intermediate value within a stated range is also included in the present application. The upper and lower limits of these smaller ranges may be independently included or excluded from the scope.

Unless otherwise stated, all technical and scientific terms used herein have the same meaning as commonly understood by those of ordinary skill in the field described in the present application. Although the present specification describes only preferred methods and materials, any methods and materials similar or equivalent to those described herein may also be used in the implementation or testing of the present specification. All literature referred to in this specification is incorporated by reference for the purpose of disclosing and describing the methods and/or materials associated with said literature. In the event of conflict with any incorporated literature, the contents of this specification shall prevail.

Without departing from the scope or spirit of the present application, a variety of improvements and modifications may be made to specific embodiments of the specification of the present application, as will be apparent to those skilled in the art. Other embodiments obtained from the specification of the present application are obvious to the skilled person. The specification and embodiments of this application are exemplary only.

As used herein, the words “comprising”, “including”, “having”, “containing”, etc., are open-ended terms, i.e. meaning including but not limited to.

### Embodiment 1 Isolation and Identification of Bacillus coagulans

Medium: liquid and solid DeMan-Rogosa-Sharpe (MRS) media (Qingdao Hope-bio), liquid and solid MC media (Hangzhou Basebio), liquid and solid brain heart infusion (BHI) media (Qingdao Hope-bio).

Salt beans of 10 grams (g) are added into a triangular flask, after 30 minutes (min) of table-shaking, 1 milliliter (mL) of the material in the triangular flask is sucked into a 9 mL diluted solution to obtained a dilution with a concentration of 10⁻¹, followed by gradient dilution of 6 times; then it is coated and inoculated onto MRS/MC/BHI agar medium in turn, followed by anaerobic inverted culture at 37 degrees Celsius (° C.) for 72 hours (h); the cultured medium are taken out, and the colonies on the solid medium are observed in terms of colony morphology, including shape, color, size, surface, edge, bulge, transparency, etc. Single bacteria with different colony morphology are selected for zoning and streaking, and cultured at 37° C. for 48 h; then the microscopic examination is carried out on the bacterial colonies, and photos are taken and recorded. The purified colonies are picked with sterile toothpicks and inoculated into liquid test tubes, followed by anaerobic culture at 37° C. for 48 h; then 1.6 mL of the liquid after 48 hours of culture is taken out and sub-packaged into a glycerol tube, followed by mixing well and storing at −80° C. after marking. Bacterial genome is extracted by bacterial genome extraction kit, and 16S ribosomal ribonucleic acid (16S rRNA) is amplified by a 27F/1492R primer and sent to Sangon Biotech for sequencing; after comparing with NCBI database, it is identified as a Bacillus coagulans.

The gene sequence of 16s rRNA is shown in the SEQ ID NO:1 as follows:

tgcggaccttttaaagcttgcttttaaaaggttagcggcggacgggtga
gtaacacgtgggcaacctgcctgtaagatcgggataacgccgggaaacc
ggggctaataccggatagttttttcctccgcatggaggaaaaaggaaag
acggcttcggctgtcacttacagatgggcccgcggcgcattagctagtt
ggtggggtaacggctcaccaaggcaacgatgcgtagccgacctgagagg
gtgatcggccacattgggactgagacacggcccaaactcctacgggagg
cagcagtagggaatcttccgcaatggacgaaagtctgacggagcaacgc
cgcgtgagtgaagaaggccttcgggtcgtaaaactctgttgccggggaa
gaacaagtgccgttcgaacagggggcgccttgacggtacccggccagaa
agccacggctaactacgtgccagcagccgcggtaatacgtaggtggcaa
gcgttgtccggaattattgggcgtaaagcgcgcgcaggcggcttcttaa
gtctgatgtgaaatcttgcggctcaaccgcaagcggtcattggaaactg
ggaggcttgagtgcagaagaggagagtggaattccacgtgtagcggtga
aatgcgtagagatgtggaggaacaccagtggcgaaggcggctctctggt
ctgtaactgacgctgaggcgcgaaagcgtggggagcaaacaggattaga
taccctggtagtccacgccgtaaacgatgagtgctaagtgttagagggt
ttccgccctttagtgctgcagctaacgc.

### Embodiment 2 Application of Bacillus coagulans BC69

### 1. Materials and Methods

### 1.1 Materials and Diet

Bacillus coagulans BC69, from Thankcome Biological Science and Technology (Suzhou) Co., Ltd. (6×10⁸ colony-forming unit (CFU/g)). Normal diet (ND, 3.8 kilocalories per gram (kcal/g)), high-sugar and high-fat diet (HSD, 4.7 kcal/g) are purchased from Beijing Botai Hongda Biotechnology Co., Ltd.

### 1.2 Experimental Animals

The experiment uses BL/6J male mice purchased from Shanghai Jiesijie Laboratory Animal Co., Ltd. All mice are placed in a controlled environment, with a 12 h daily light/dark cycle, maintained room temperature of 22±2° C. and humidity of 50%-60% in the experimental room, and free access to food and water. After one week of adaptation, the mice are randomly divided into 4 groups, with 8 mice in each group, and fed for 16 weeks. The specific experiment is lasted from Nov. 28, 2020 to Mar. 25, 2021. Each group is named according to the feed and the gavage substances they are fed, see Table 1 for nomenclature and abbreviations. No significance notation is omitted as there is no difference between gavage and non-gavage groups in this section. The HSD+BC69-1 group is a probiotic prophylactic group with daily gavage of Bacillus coagulans BC69 from the beginning of the experiment; the HSD+BC69-2 group is a treatment group subjecting to gavage treatment from the 11^{th} week. Daily dosage of Bacillus coagulans BC69: gavage dose of 3×10⁵ CFU/g, where a mouse of 20 g is given 0.4 mL of 1.5×10⁷ CFU/mL diluted solution of Bacillus coagulans BC69 ((1 g bacterial powder diluted in 40 mL normal saline).

TABLE 1
Experimental grouping

| | | Abbreviation of |
|---|---|---|
| Feed | Gavage substance | group name |
| Conventional feed | Normal saline | NFD |
| High sugar and high fat diet | Normal saline | HSD |
| High sugar and high fat diet | Bacillus coagulans BC69 | HSD + BC69-1 |
| High sugar and high fat diet | Normal saline for the first 10 weeks and | HSD + BC69-2 |
| | Bacillus coagulans BC69 for the last 6 | |
| | weeks | |

### 1.3 Biochemical Analysis

Blood sugar level is measured by Roche blood glucose meter, serum triglyceride (TG), total cholesterol (TC), high-density lipoprotein cholesterol (HDL-C) and low-density lipoprotein cholesterol (LDL-C) are measured by kits of Nanjing Jiancheng Bioengineering Institute, and tumor necrosis factor (TNF-α) and interleukin-6 (IL-6) protein levels are also detected by kits of Nanjing Jiancheng Bioengineering Institute; the measurement is carried out in accordance with the manufacturer's instructions.

### 1.4 Histological Analysis

The liver, epididymal fat and perirenal fat are fixed with 4% paraformaldehyde, embedded in paraffin, sectioned into a thickness of 4 micrometers (m) and stained with hematoxylin-eosin (HE). The liver is embedded with OCT cryo-embedding agent, sectioned into a thickness of 8 m and stained with oil red O. The stained sections are observed under a Nikon Eclipse E100 light microscope.

### 1.5 Oral Glucose Tolerance Test (OGTT)

The OGTT is conducted after 12 hours of fasting and mice are given glucose standard solution (200 grams per liter, g/L) by gavage at 2 gram per kilogram (g/kg) (glucose/body weight), a drop of peripheral blood is collected from the tail vein and blood glucose levels are measured by glucometer at 0, 15, 30, 60, 90 and 120 min after glucose gavage.

### 1.6 Analysis of Intestinal Flora

Genomic deoxyribonucleic acid (DNA) is extracted from fecal samples taken from colon during dissection by AxyPrepDNA Kit (AXYGEN Company). Universal primers 343F-806R (5′-TACGGRAGGCAGCAG-3′ (SEQ ID NO:2), 5′-GGACTACHVGGGTWTCTAAT-3′(SEQ ID NO:3)) for V3-V4 region of 16S rRNA of prokaryotic rRNA are subjected to polymerase chain reaction (PCR) amplification, with amplification conditions of: pre-denaturation at 94° C. for 5 min, denaturation at 95° C. for 30 seconds (s), annealing at 56° C. for 30 s, extension at 68° C. for 45 s, a total of 35 cycles, extension at 68° C. for 5 min. After purification, the amplified products are detected and quantified by QuantiFluor™-ST blue fluorescence quantitative system (Promega company), then the Miseq library is constructed and sequenced on a machine. The sequences obtained by Miseq sequencing are spliced, and after quality control and filtration, the samples are analyzed by operational taxonomic units (OTUs) cluster analysis and species taxonomy.

Mothur 1.30.2 software is used to calculate the estimation of alpha (α) diversity, and Qiime 1.9.1 software is used to generate the abundance table of each taxonomic level and calculate the Beta diversity distance. UPGMA is used to cluster the samples hierarchically. The principal coordinate analysis of unweighted UniFrac distance matrix is carried out by using R software. Based on the data in Kyoto Encyclopedia of Genes and Genomes (KEGG) pathway database, PICRUSt is used to predict the function of microbial community.

### 1.7 Analysis of Short-Chain Fatty Acids (SCFAs)

The derivatized SCFAs (including acetic acid, propionic acid and butyric acid) in feces are analyzed by gas chromatography-mass spectrometry.

### 1.8 Data Analysis

The data are expressed by mean±SEM. SPSS 26 software is used for statistical analysis. The statistical difference is analyzed by one-way analysis of variance (ANOVA), with P<0.05 indicating statistical difference. Difference with normal feed diet (NFD) is indicated by #(#means P<0.05, ##means P<0.01, ###means P<0.001), difference with HSD is indicated by * (* means P<0.05, ** means P<0.01, *** means P<0.001), and ns stands for no significance.

### 2. Results and Discussion

### 2.1 Weight Changes

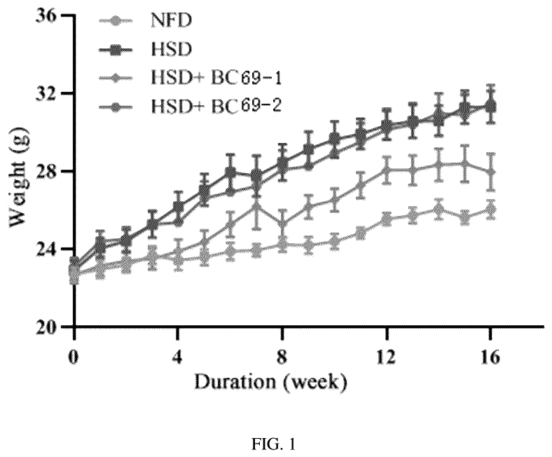

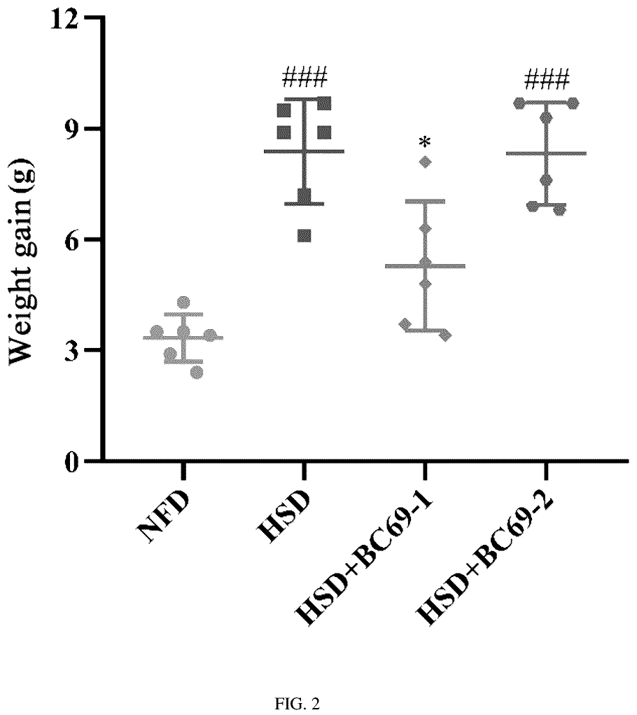

As can be seen from FIG. 1 and FIG. 2, there is a consistent weight gaining and a highly significant difference in the amount of weight gaining throughout the cycle in the HSD group compared to the NFD group (p<0.001). The weight gaining in the HSD+BC69-1 group is slower than that of the HSD group and differed in the amount of increase compared to that of the HSD group (p<0.05), and higher than that of the NFD group yet with no statistical difference; the weight gaining in the HSD+BC69-2 group is similar to that of the HSD group and is highly significant compared to that of the NFD group (p<0.001), suggesting that prophylactic gavage of Bacillus coagulans BC69 at 3×10⁵ CFU/g is able to alleviate the weight gaining induced by high sugar and high fat diets in mice.

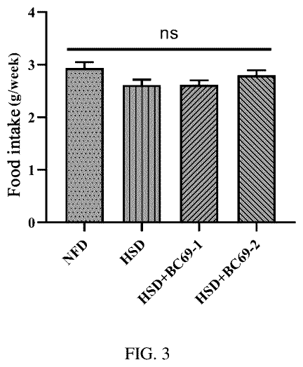

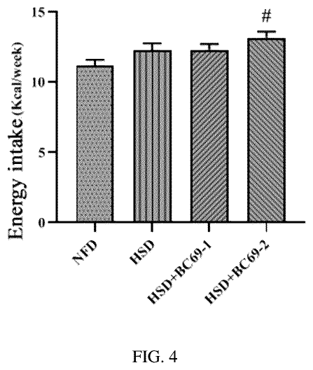

The results of food intake and energy intake for each group are shown in FIG. 3-FIG. 4, which suggest that the energy intake of the HSD and HSD+BC69-1 groups is slightly higher than that of the NFD group, and the energy intake of the HSD+BC69-2 group is significantly higher than that of the NFD group. The energy intake of the HSD+BC69-1 group is basically the same as that of the HSD group, with no statistical difference, suggesting that the reduction in body weight in the HSD+BC69-1 group is not related to the reduction in the food intake and energy intake compared to the HSD group.

### 2.2 Inflammatory Factors

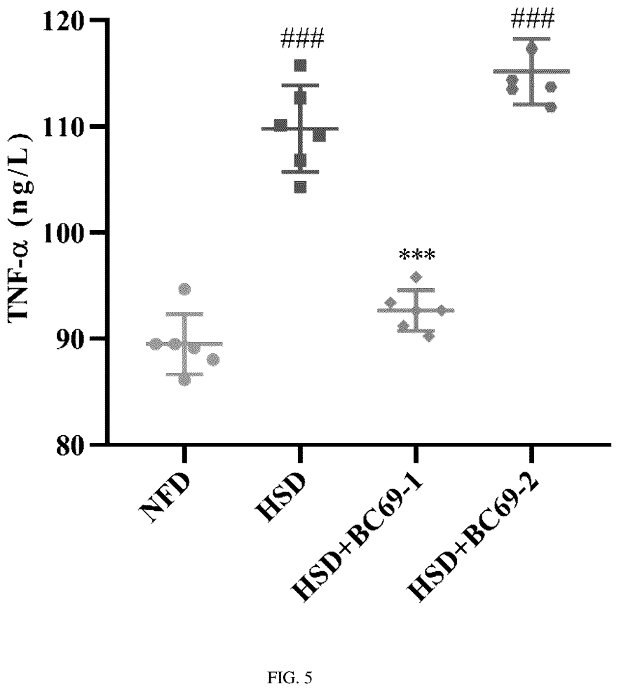

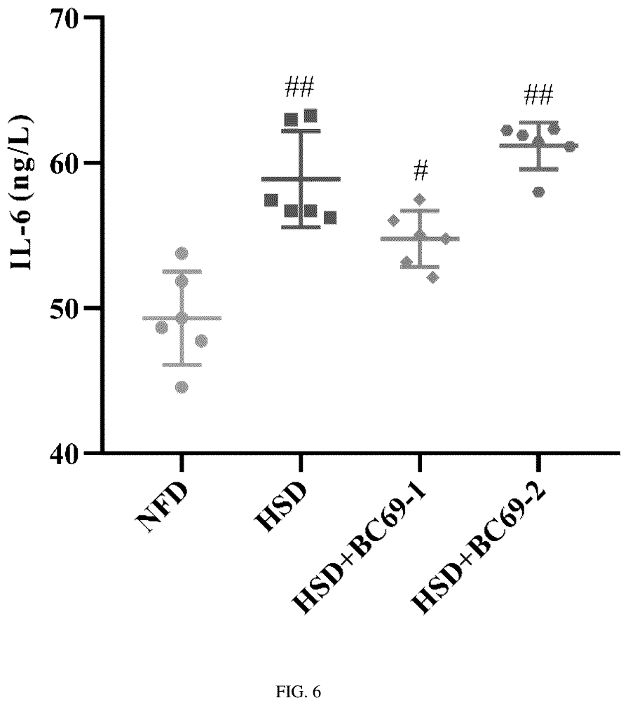

The determination results of TNF-α and IL-6 in the serum of the present application are shown in FIG. 5 and FIG. 6. It can be seen from the drawings that both TNF-α and IL-6 are increased in the HSD group compared to those in the NFD group, with significant differences (p<0.01), indicating that continuous intake of high sugar and high fat diets induces an inflammatory response in the organism. There is a highly significant decrease of TNF-α in the HSD+BC69-1 group compared to that of the HSD group (p<0.001) and no statistically significant difference compared to that of the NFD group; TNF-α level in the HSD+BC69-2 group is not statistically different from that in the HSD and is dramatically increased compared to that in the NFD group (p<0.001). IL-6 in the HSD+BC69-1 group is decreased compared to that in the HSD group, but is not statistically different, and IL-6 in the HSD+BC69-1 group is increased compared to that in the NFD group with a difference (p<0.05); IL-6 level in the HSD+BC69-2 group is not statistically different from that in the HSD group, and is increased and significantly different in the HSD+BC69-2 group compared to that in the NFD group (p<0.01). The above results indicate that long-term intake of high sugar and high fat diets causes inflammatory reactions in mice, and that gavage with 3×10⁵ CFU/g of Bacillus coagulans BC69 provides a preventive but not curative effect on inflammatory reactions caused by high sugar and high fat diets.

### 2.3 Tissue Quality and Morphology

To investigate the effect of Bacillus coagulans BC69 intake on mouse tissues, mice are subjected to euthanasia at the end of the experimental cycle; after dissection, the wet weights of epididymal fat, perirenal fat and liver tissue (liver) are measured and recorded; meanwhile, the aforementioned tissues are fixed in paraformaldehyde and stained for analysis; epididymal fat and liver tissue are prepared into paraffin sections and subjected to HE staining, and liver tissue is prepared into frozen sections and subjected to oil red O staining.

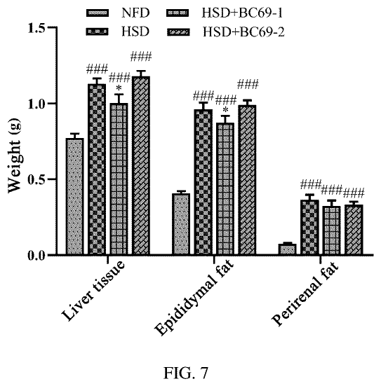

As illustrated in FIG. 7, the liver, epinephric fat and perirenal fat of the HSD group are all increased in weight compared to those in the NFD group with a highly significant difference (p<0.001); the weight of liver and perirenal fat in the HSD+BC69-1 group is reduced with highly significant difference as comparing to that of the HSD group (p<0.05), and the weight of perirenal fat is reduced in a certain amount but not statistically different; the weight of each tissue in the HSD+BC69-2 group is not statistically different from that in the HSD group. The results indicate that a high-fat diet leads to a significant increase in the weight of liver, epididymal fat and perirenal fat, and that 3×10⁵ CFU/g of Bacillus coagulans BC69 gavage provides a significant preventive effect on the weight gaining of liver and epididymal fat.

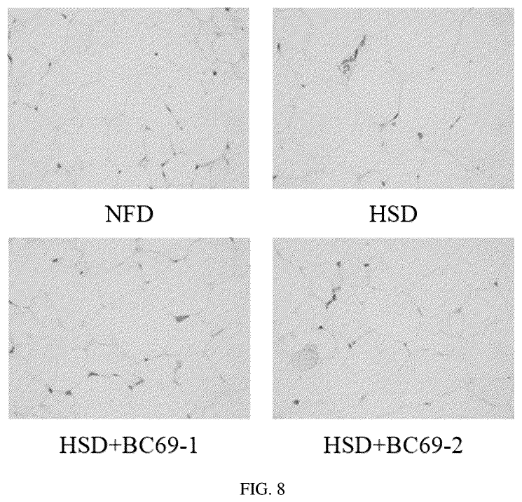

The results of HE staining of epididymal fat in mice are illustrated in FIG. 8, where larger adipocytes indicate increased accumulation of fat, and the size of the general adipocytes correlates positively with the above-mentioned epididymal fat weight. As can be seen from the FIG. 8, the HSD group shows significant balloon-like changes compared to the NFD group; the area of adipocytes undergoing balloon-like changes become smaller in the HSD+BC69-1 and HSD+BC69-2 groups compared to that of the HSD group, and the result of the HSD+BC69-1 group is better than that of the HSD+BC69-2 group. The above results suggest that continuous intake of high sugar and high fat diet causes the adipocyte area of mice to increase, while continuous prophylactic gavage of Bacillus coagulans BC69 on the basis of high sugar and high fat diet can reduce the adipocyte area of mouse epididymis fat to a certain extent and reduce the fat accumulation in mouse, indicating that 3×10⁵ CFU/g of Bacillus coagulans BC69 gavage is capable of reducing the accumulation of epididymis fat in mouse.

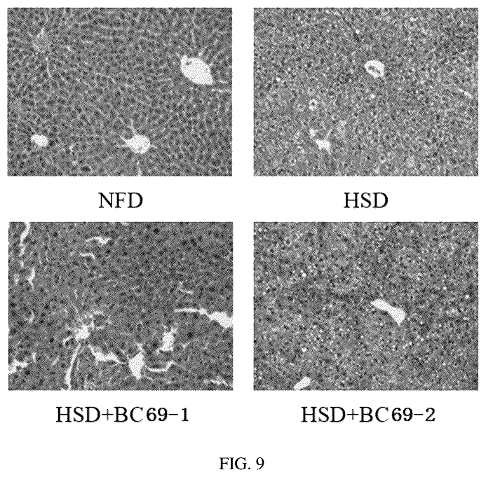

FIG. 9 shows the results of HE staining in mouse liver tissue, which demonstrate that the normal group (NFD group) has neatly arranged cells and clear hepatic cords, with no significant inflammatory cell infiltration or inflammatory foci. Compared to the NFD group, the model group (HSD group) shows crowding of nuclei to one side, bubble-like enlargement of hepatocytes, irregular arrangement of cells, unclear hepatic cords and more vacuoles (fat droplets) in the cytoplasm, while the number of vacuoles in the HSD+BC69-1 group is reduced compared to that in the HSD group, and there is no significant change in the HSD+BC69-2 group.

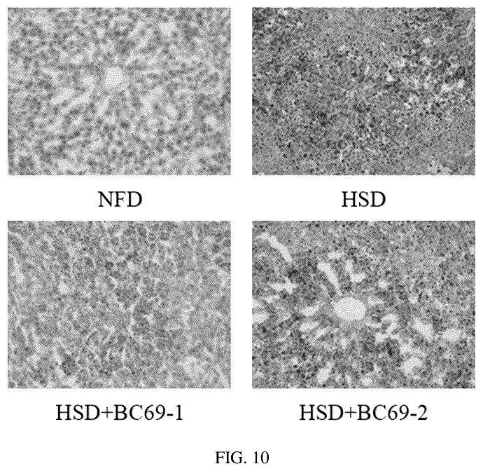

The results of oil red O staining of mouse liver tissue are shown in FIG. 10, in which the area stained red is positively proportional to the degree of fat accumulation, and it is evident as shown in FIG. 10 that the area stained red is significantly greater in the HSD group compared to that in the NFD group, suggesting serious fat accumulation in the liver; the red areas in the HSD+BC69-1 and HSD+BC69-2 groups are reduced compared to that in the HSD group, with the reduction in the HSD+BC69-1 group being more apparent; taken together, the results demonstrate that continuous intake of high sugar and high fat diet can aggravate fat accumulation in the liver, and prophylactic gavage of Bacillus coagulans BC69 at 3×10⁵ CFU/g is effective in alleviating the accumulation of liver fat in mice fed with high sugar and high fat diets.

### 2.4 Influence on Memory Ability

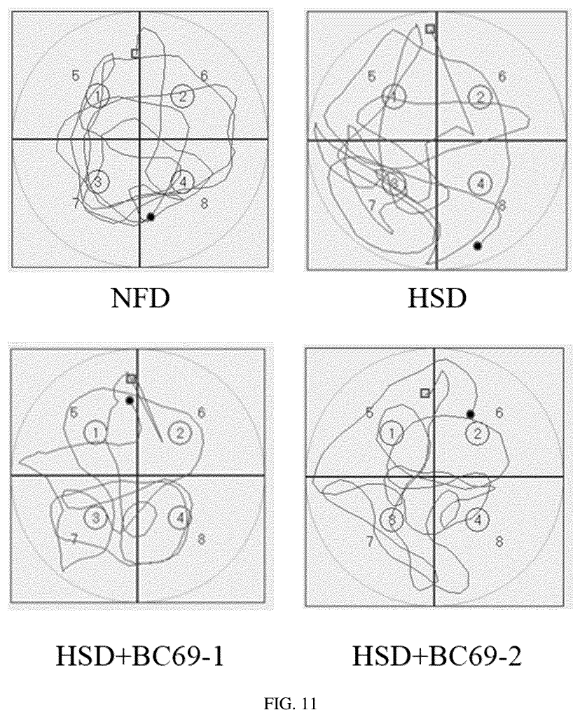

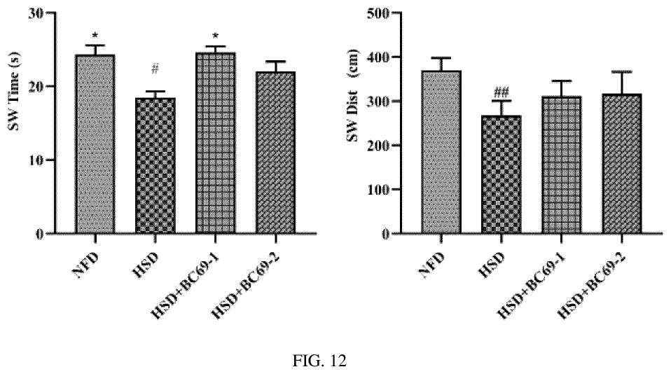

To investigate the effect of Bacillus coagulans BC69 intake on the memory ability of mice, a mice water maze experiment is conducted before the end of the experimental cycle and the results are shown in FIG. 11 and FIG. 12. The distance and time spent in the target quadrant are reduced with significant difference in the HSD group compared to those in the NFD group (p<0.05), and the time spent of moving in the target quadrant is significantly increased in the HSD+BC69-group compared to that in the HSD group (p<0.05). These results suggest that the intake of high sugar and high fat diets significantly impairs memory ability and that 3×10⁵ CFU/g of Bacillus coagulans BC69 by gavage has a mitigating effect on this reduced memory ability.

### 2.5 Influence on SCFAs

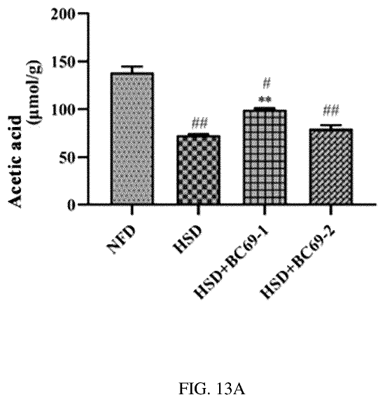

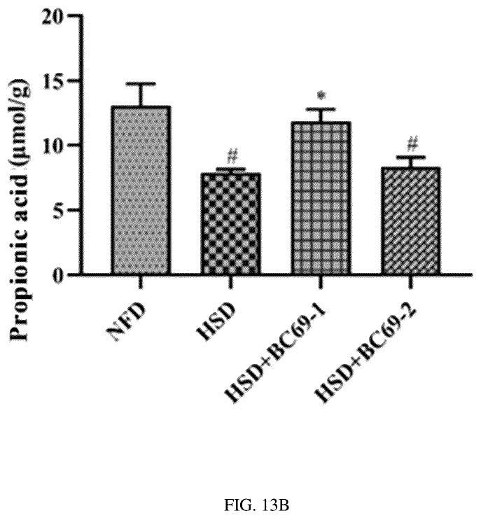

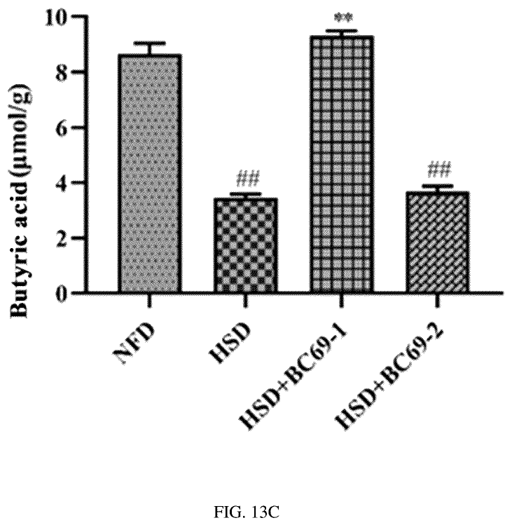

The SCFAs of four groups of mice, NFD, HSD, HSD+BC69-1 and HSD+BC69-2, are measured in fecal samples taken at the end of the experimental cycle and the results are shown in FIG. 13A, FIG. 13B and FIG. 13C. As can be seen from the FIG. 13A, the contents of acetic acid are decreased in the HSD and HSD+BC69-2 groups compared to that in the NFD group with a significant difference (p<0.01) and decreased in the HSD+BC69-1 group with a statistically significant difference (p<0.05); the acetic acid is increased in the HSD+BC69-1 group compared to that in the HSD group with a significant difference (p<0.05), and no significant change is observed in the HSD+BC69-2 group. As can be seen from FIG. 13B, the propionic acid is decreased in both the HSD and HSD+BC69-2 groups compared to that of the NFD group with statistical difference (p<0.05), while the propionic acid is decreased but not statistically different in the HSD+BC69-1 group as compared with that of the HSD group, and the propionic acid in HSD+BC69-2 group shows no significant difference; FIG. 13C shows that the butyric acid is decreased and significantly different (p<0.01) in both HSD and HSD+BC69-2 groups compared to that in the NFD group, and there is no significant difference of butyric acid in the HSD+BC69-1 group; compared to the HSD group, butyric acid is significantly increased with significant difference (p<0.01) in the HSD+BC69-1 group, yet there is no significant difference of butyric acid in the HSD+BC69-2 group. The above results suggest that SCFAs in the feces of mice are reduced when mice are fed chronically with high sugar and high fat diets, and that 3×10⁵ CFU/g of Bacillus coagulans BC69 by gavage has a significant preventive but not curative effect on the reduction of SCFAs, especially butyric acid.

### 2.6 Influence on Intestinal Flora

### 2.6.1 OTUs and Alpha-Diversity Analysis of Intestinal Microflora

To investigate the effect of high sugar and high fat diet and Bacillus coagulans BC69 on the intestinal flora of mice, three randomly selected mouse fecal samples from each of the four groups NFD, HSD, HSD+BC69-1 and HSD+BC69-2 are sequenced for their intestinal flora. 589 OTUs, including 11 phyla, 87 families and 177 genera, are identified in the 12 samples selected, and 337 OUTs are common to the four groups according to the Venn diagram.

### 2.6.2 Hierarchical Clustering and Principal Co-Ordinates Analysis (PCoA)

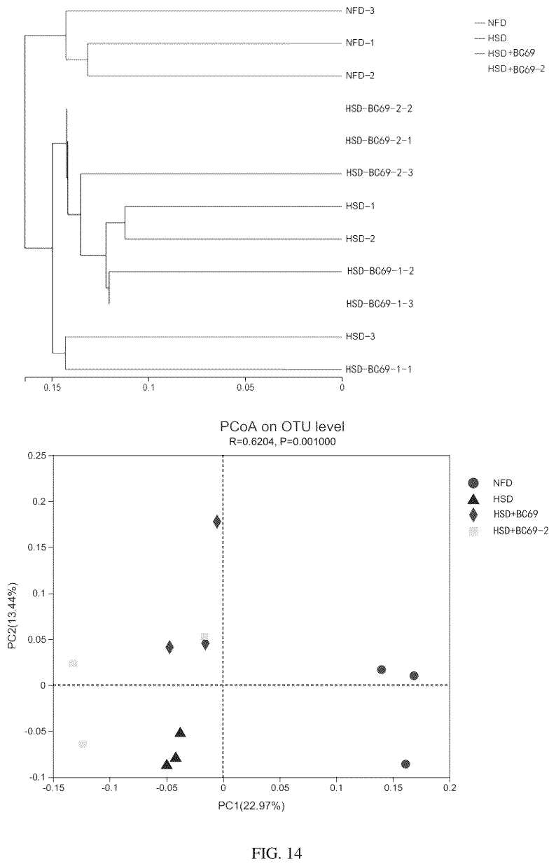

To investigate the effects of high sugar and high fat diet and Bacillus coagulans BC69 on the structure of the intestinal flora of mice, the OTUs obtained from sequencing are subjected to hierarchical clustering and PCoA, and the results are shown in FIG. 14. From the graph of the results of the hierarchical cluster analysis at the classification level of OUTs, it is observed that the intake of high sugar and high fat diet and Bacillus coagulans BC69 has changed the overall structure of the intestinal flora; PCoA of the OTUs abundance matrix derived by the Unweighted UniFrac distance algorithm show that there is a significant difference in the beta (β)-diversity of the intestinal microbial community between the four groups of samples, indicating that the composition of the intestinal flora is significantly changed, with the HSD and HSD+BC69-1 groups differing in the dimension of PC2, and HSD+BC69-2 varying between the two in this dimension.

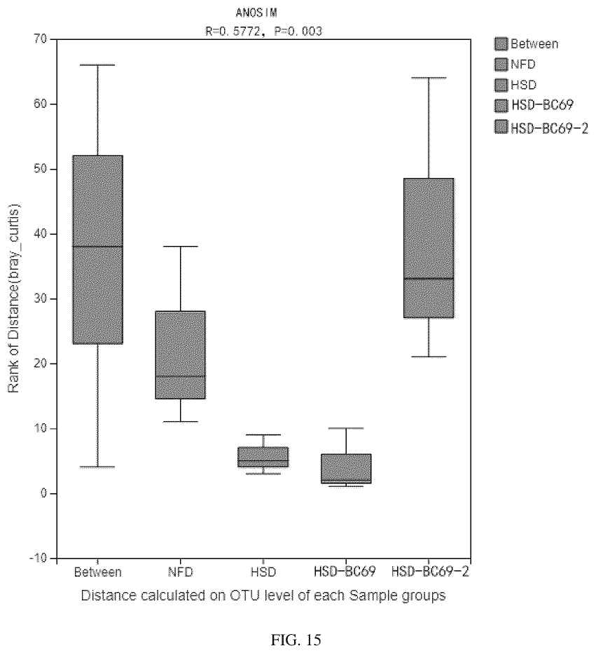

Analysis of similarities (ANOSIM) is a non-parametric test used to test whether differences between groups are significant, i.e. whether they are greater than differences within groups, and thus whether the grouping is meaningful. The distance between each two samples is calculated based on the Bray-Curtis algorithm. As shown in FIG. 15, the results of the ANOSIM suggest that the differences within the HSD and HSD+BC69-1 groups are relatively small and the grouping is reasonable (ANOSIM, R=0.5772, P=0.003).

### 2.6.3 Species Composition Analysis of Mouse Fecal Flora

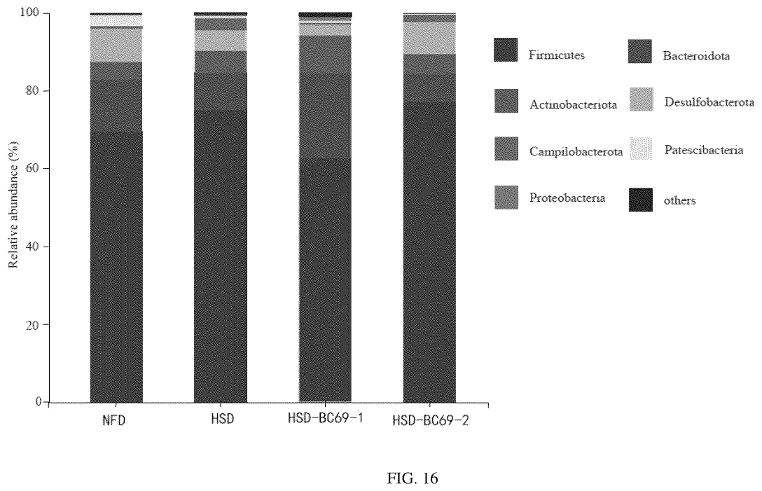

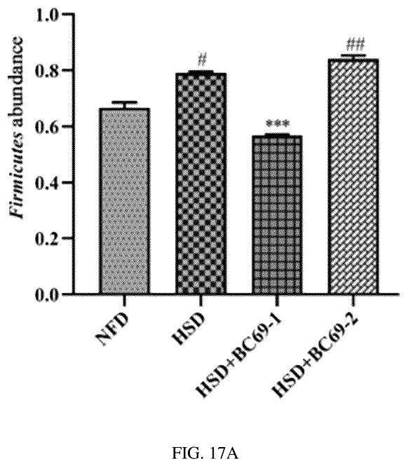

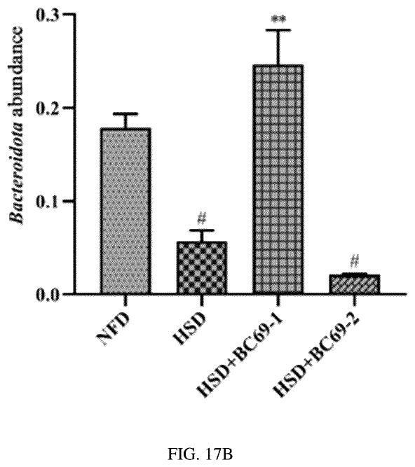

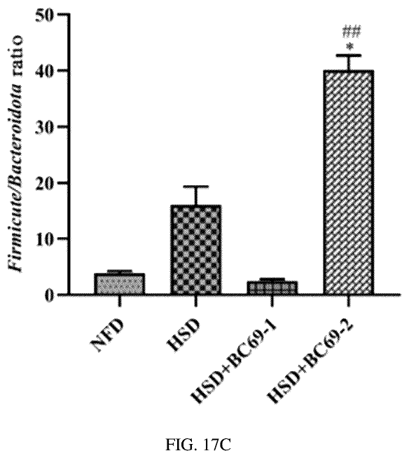

The distribution of bacterial flora abundance at the phylum level in each group of mouse fecal samples is shown in FIG. 16, and it can be seen form the FIG. 16 that the percentage of each flora varies in the feces of each mouse, with the dominant flora mainly being Firmicutes and Bacteroidota, while Actinobacteriota, Desulfobacterota and Campilobacterota account for a relatively low percentage. FIG. 17A, FIG. 17B and FIG. 17C show that the ratio of Firmicutes/Bacteroidota of mice in the model group fed high sugar and high fat diet is also increased compared to that of normal control group. The percentage of Firmicute is significantly reduced and the percentage of Bacteroidota is increased by the prophylactic gavage of Bacillus coagulans BC69, resulting in an alleviated increasing of Firmicute/Bacteroidota ratio caused by the feeding of high sugar and high fat diets, as well as a regulatory effect on the restore of the intestinal flora to a normal level. Therefore, it is concluded that Bacillus coagulans BC69-1 is effective in improving the dysbiosis of the intestinal flora caused by a high sugar and fat diet through its preventive effect.

### 2.6.3.2 Species Composition Analysis of Intestinal Flora in Mice (Family Level)

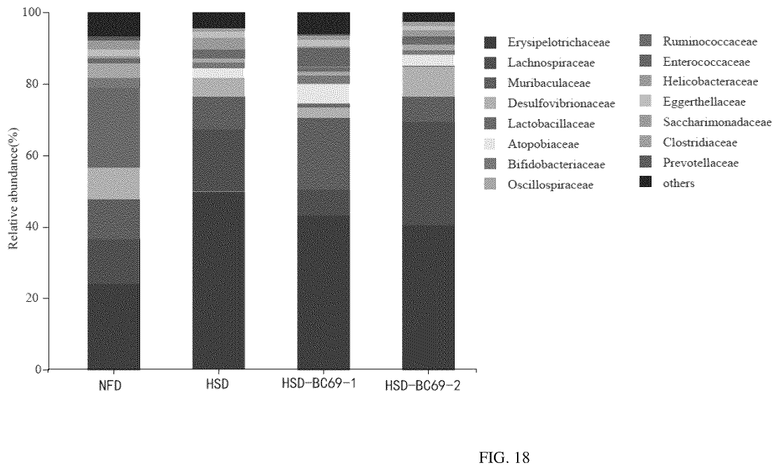

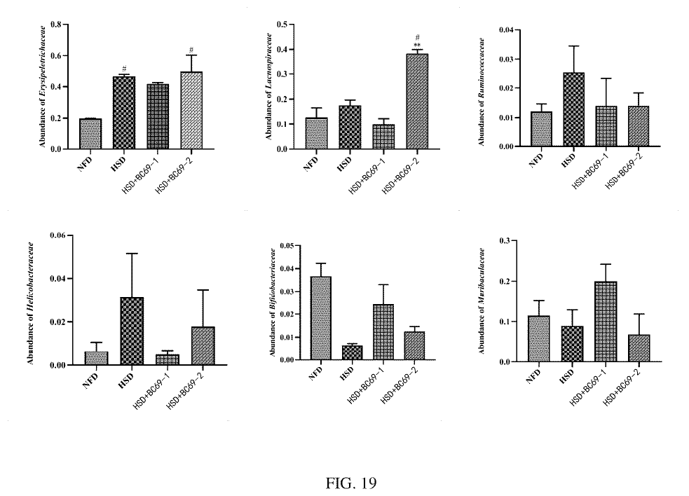

FIG. 18 and FIG. 19 show the distribution of bacterial flora abundance at the family level in mouse feces samples of each group, where the dominant flora (>5% in all groups) are mainly Erysipelotrichaceae, Muribaculaceae and Lachnospiraceae, with a lower proportion of Bifidobacteriaceae, Ruminococcaceae, Helicobacteraceae, Atopobiaceae, Lactobacillaceae and Prevotellaceae.

Of these, high sugar and fat diets cause increases in Erysipelotrichaceae, Lachnospiraceae, Ruminococcaceae and Helicobacteraceae, and prophylactic gavage by Bacillus coagulans BC69 ameliorates the increases caused by high sugar and fat diets; high sugar and fat diet leads to decreases in Bifidobacteriaceae and Muribaculaceae, and prophylactic gavage by Bacillus coagulans BC69 ameliorates the decreases.

### 2.6.3.3 Species Composition Analysis of Intestinal Flora in Mice (Genus Level)

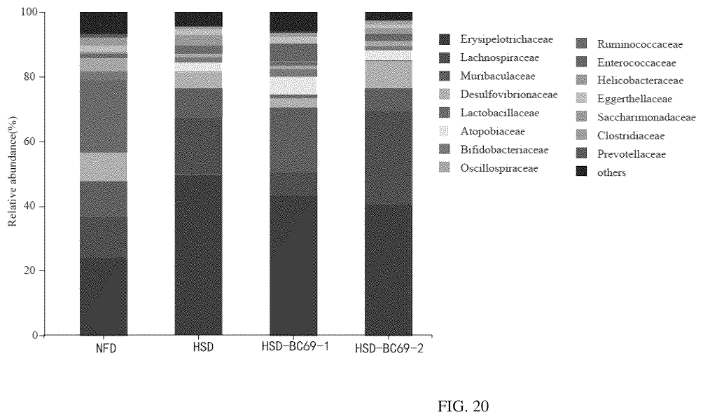

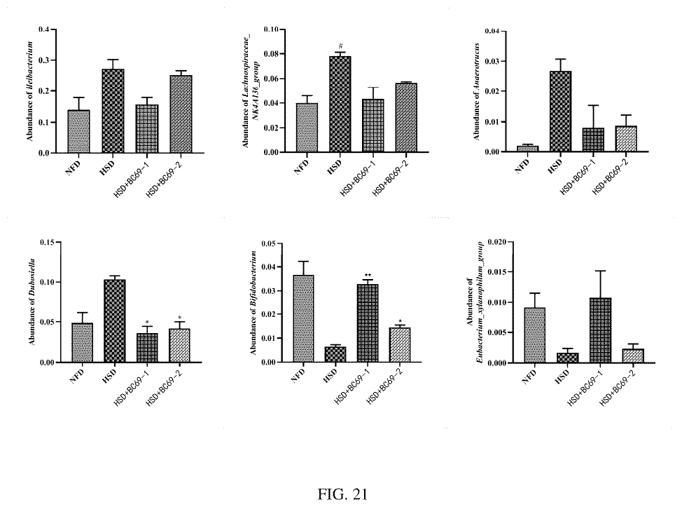

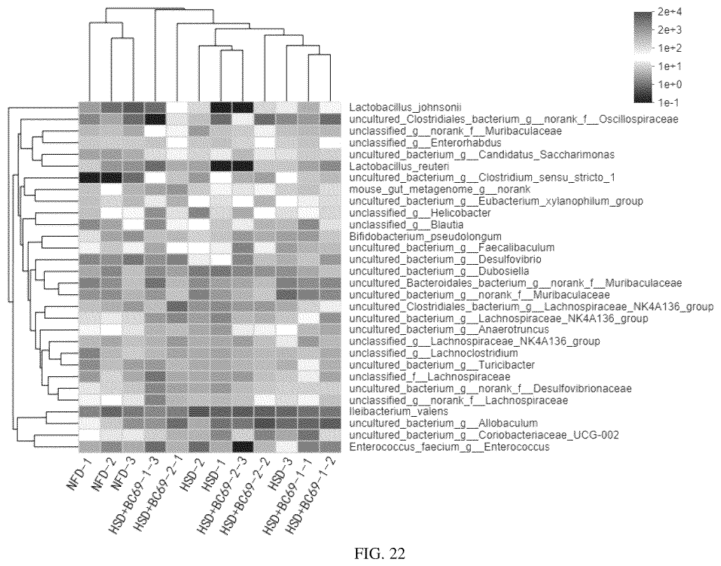

FIG. 20-FIG. 22 illustrate the distribution of bacterial flora abundance at the genus level for each group of mice fecal samples; the predominant flora in the feces of each mouse are Ileibacterium, Allobaculum, Anaerotrucus, Lachnospiraceae_NK4A136_group, Dubosiella, Bifidobacterium, and Eubacterium_xylanophilum_group, etc. Among them, high sugar and high fat diets cause an increase in Ileibacterium, Anaerotrucus, Dubosiella and Lachnospiraceae_NK4A136_group, and prophylactic gavage by Bacillus coagulans BC69 can ameliorate the increase caused by high sugar and high fat diets; high-sugar and high-fat diets reduce the Bifidobacterium and Eubacterium_xylanophilum_group, and prophylactic gavage with Bacillus coagulans BC69 ameliorates the reduction caused by high-sugar and high-fat diets.

### 2.6.4 Functional Prediction Analysis

### 2.6.4.1 Functional Prediction of Phylogenetic Investigation of Communities by Reconstruction of Unobserved States (PICRUST) 2

PICRUSt is a software package for functional prediction of 16S amplicon sequencing results. Firstly, the OTUs abundance table is normalized by PICRUSt (the PICRUSt process stores the clusters of orthologous groups (COG) information and KEGG Ortholog (KO) information corresponding to the greengene id), i.e. the effect of the number of copies of the 16S marker gene in the species genome is removed; then the COG family information and KO information corresponding to the OTUs are obtained by the greengene id corresponding to each OTU; and the abundance of each COG and KO abundance are calculated. Based on the information from the COG database, the descriptive information of each COG can be parsed from the eggNOG database, as well as its functional information to obtain a functional abundance profile; based on the information from the KEGG database, KO, Pathway and Enzyme pathway information may be obtained, and the abundance of each functional category may be calculated based on OTU abundance. Moreover, PICRUSt may be used to obtain information on the 3 levels of metabolic pathways for Pathway and obtain the abundance table for each level separately.

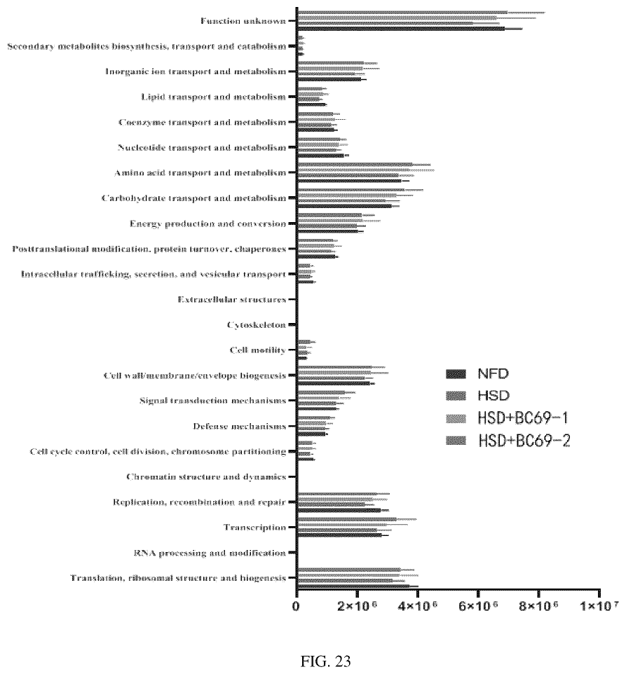

As shown in FIG. 23, the results of predicting the metabolic pathway of the KEGG module using 16S rRNA data from PICRUSt2 (n=3 per group) indicate that the effect of supplementing Bacillus coagulans BC69-1 on the functional potential of the intestinal microbiome is demonstrated in HSD-fed mice, with the metabolic pathway behaving more like the NFD as a result of supplementing Bacillus coagulans BC69-1 under HSD feeding.

### 2.6.4.2 Phenotypic Prediction of Bugbase

BugBase (https://bugbase.cs.umn.edu/index.html) is a microbiome analysis tool that identifies high level phenotypes present in microbiome samples and is able to perform phenotype prediction. Firstly, the BugBase normalises the OTU by the predicted 16S copy number and then uses the pre-computed file provided to predict the microbial phenotypes, and there are seven major phenotypes including Gram Positive, Gram Negative, Biofilm Forming, Pathogenic, Mobile Element Containing, Oxygen Utilizing (including Aerobic, Anaerobic, facultatively anaerobic) and Oxidative Stress Tolerant.

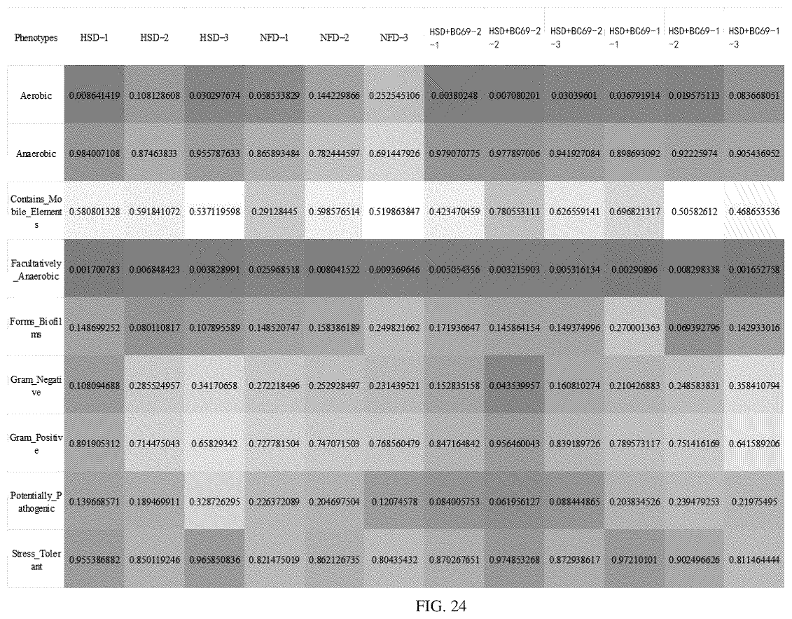

According to the BugBase-based phenotypic predictions as shown in FIG. 24, all samples are dominated by anaerobic, gram-positive bacteria, and the HSD+BC69-1 group has the lowest potential pathogenicity, the best performance on the dimension of potential pathogenicity.

The embodiments described above describe only the preferred way of the present application and are not intended to limit the scope of the present application. Without departing from the spirit of the design of the present application, all variations and improvements made to the technical schemes of the present application by persons of ordinary skill in the art shall fall within the scope of protection determined by the claims of the present application.

What is claimed is:

1. A medication with an efficacy of relieving hyperglycemia and hyperlipidemia, wherein the medication comprises Bacillus coagulans or secondary metabolites of the Bacillus coagulans as main active ingredients, wherein the Bacillus coagulans is preserved in China General Microbiological Culture Collection Center (CGMCC), Institute of Microbiology Chinese Academy of Sciences on Nov. 10, 2021, with a preservation number of CGMCC No. 23766 and a preservation address of Institute of Microbiology, Chinese Academy of Sciences, No. 3, No. 1 Beichen West Road, Chaoyang District, Beijing; and
the Bacillus coagulans has a bacterial content of 3×10⁵ CFU/g.

2. An application of a Bacillus coagulans in preparing a medication with an efficacy of relieving hyperglycemia and hyperlipidemia, wherein the medication has any one of functions as follows:
(1) reducing weight increased by high sugar and high fat;
(2) preventing inflammatory reaction caused by high sugar and high fat;
(3) preventing fat weight increasing in liver and epididymal fat caused by high sugar and high fat;
(4) alleviating declining of memory ability caused by high sugar and high fat;
(5) preventing short-chain fatty acids decreasing caused by high sugar and high fat; and
(6) improving dysbiosis of intestinal flora caused by high sugar and high fat;
the Bacillus coagulans is preserved in China General Microbiological Culture Collection Center, Institute of Microbiology Chinese Academy of Sciences on Nov. 10, 2021, with a preservation number of CGMCC No. 23766 and a preservation address of Institute of Microbiology, Chinese Academy of Sciences, No. 3, No. 1 Beichen West Road, Chaoyang District, Beijing.
